(19) United States
(12) Patent Application Publication
(10) Pub. No.: US 2025/0170334 A1
(43) Pub. Date: May 29, 2025

(54) REDUCED FRICTION OIL-FREE SYRINGE PLUNGER FEEDING TUBES AND METHODS OF USING THE SAME FOR FEEDING PLUNGERS INTO SYRINGES

(71) Applicant: SiO2 Medical Products, Inc., Auburn, AL (US)

(72) Inventors: Matteo Trisoglio, Waldkirch (DE); Ralf Kibele, Bruckmuehl (DE); Ahmad Taha, Auburn, AL (US)

(21) Appl. No.: 18/842,658

(22) PCT Filed: Mar. 3, 2023
(86) PCT No.: PCT/US2023/063685

§ 371 Date: Aug. 29, 2024

Related U.S. Application Data
(60) Provisional application No. 63/268,872, filed on Mar. 4, 2022.

(51) Int. Cl. A61M 5/31

(52) U.S. Cl. CPC A61M 5/3129; A61M 2005/3131; A61M 2205/0238; A61M 2207/00; A61M 2207/10

(57) ABSTRACT

Disclosed are feeding tubes for placing syringe gaskets into medical barrels and related systems and methods of using such feeding tubes. An optional tube according to the disclosure is configured to retain and feed a syringe gasket into a medical barrel. The tube includes an elongate hollow tubular body comprising an internal lumen. The lumen has an inner wall which consists of a contact surface configured to contact a portion of the circumferential surface portion of the gasket and a non-contact surface occupying the remainder of the inner wall.

### CROSS-REFERENCE TO RELATED APPLICATIONS

This application claims priority to U.S. Provisional Patent Application No. 63/268,872, entitled “REDUCED FRICTION OIL-FREE SYRINGE PLUNGER FEEDING TUBES AND METHODS OF USING THE SAME FOR FEEDING PLUNGERS INTO SYRINGES,” filed on Mar. 4, 2022, the contents of which are incorporated by reference herein in their entirety.

### BACKGROUND OF THE INVENTION

### 1. Field of Invention

The disclosed concept relates to apparatus and components thereof for feeding plungers into syringe barrels. More particularly, the disclosed concept relates to plunger feeding tube configurations that provide reduced frictional resistance to facilitate feeding of plungers into syringes without a flowable lubricant.

### 2. Description of Related Art

There are three traditional methods for assembling a conventional syringe plunger, i.e., gasket, into a prefilled syringe. The first is use of a vent tube, wherein the gasket is pushed through a tube that is placed into the syringe and exits out the bottom of the tube into its final position within the syringe barrel. The second is use of a vacuum, which is created in the syringe and the gasket is introduced into the opening thereof. Differential pressure forces the gasket down into the barrel into a final position. A third method, known as vacuum assist, creates a vacuum and further includes a mechanical element to assist placement of the gasket into its final position.

Regardless of which method is used, feeding a gasket into a syringe involves use of a tube into which a gasket is inserted and from which the gasket is fed into the proximal open end of a medical barrel, e.g., syringe barrel. Such a “feeding tube” is typically made from metal and must be configured to retain the gasket in place when being positioned above the open end of the syringe barrel while at the same time allowing the gasket to advance with relatively low frictional resistance within and from the feeding tube. High friction could damage the gasket, position it improperly or slow down the in-line feeding process.

For background and to illustrate the state of the art, FIG. 6 shows a prefilled syringe 1010 comprising a medical barrel 1012 into which a liquid drug product 1014 is filled. The medical barrel 1012 includes a proximal open end 1018 into which a feeding tube 1020 is inserted in order to deposit a gasket 1016 into the medical barrel 1012. As shown, the gasket 1016 serves to seal the liquid drug product 1014 within the medical barrel 1012. When it is time to administer the drug product 1014 to a patient, the gasket 1016 is advanced in a distal direction, e.g., with a plunger rod that is coupled to the gasket 1016 and which protrudes out from the proximal open end 1018, in order to dispense the drug product 1014.

As shown in FIG. 7, the feeding tube 1020 has circular internal and external geometry. The feeding tube 1020 includes a hollow tubular body 1022 having an inner wall 1026 defining a lumen 1024. The lumen 1024 is configured to have a gasket placed therein, prior to feeding the gasket into a syringe barrel. The inner wall 1026 includes a contact surface 1028 configured to contact the outer wall of a gasket disposed within the lumen 1024. Notably, the inner wall 1026 and contact surface 1028 of this prior art feeding tube 1020 are one and the same. In other words, the inner wall 1026 and contact surface 1028 are coextensive. As such, the outer wall of a gasket. at least along the widest section(s) of one or more cylindrical portions thereof, contacts the entirety of the inner wall 1026 when disposed in and advanced down the lumen 1024. Applicant has determined that such full surface contact, which traditional feeding tubes provide, is at least partially responsible for elevated frictional resistance when the gasket is advanced down the lumen 1024.

To address the friction issue, liquid or gel-like flowable lubricants, such as free silicone oil (e.g., polydimethylsiloxane or “PDMS”), are typically used to provide lubrication between the gasket and the inner surface of the feeding tube to provide desirably low frictional resistance. For example, the inner surface of the feeding tube may be spray coated with silicone oil. Silicone oil is, in fact, a standard flowable lubricant used in the industry. However, silicone oil within a feeding tube will transfer onto the gasket surface during feeding and will consequently be found inside a filled syringe as oil droplets, For some applications, this can be problematic. One reason is that a flowable lubricant can mix and interact with the drug product in a syringe, potentially degrading the drug or otherwise affecting its efficacy and/or safety. Degradation is particularly problematic in the case of protein compositions and polypeptide compositions. Some biopharmaceutical products are susceptible to one or more negative effects from interaction with particles generated from a flowable lubricant. Such negative effects may include denaturing of proteins, agglomeration of proteins, degradation of proteins, triggering an undesired immune response in a patient who is administered the drug product and degrading efficacy of the drug product. Moreover, effects on the drug product aside, the lubricants themselves may present a health risk when injected into a patient. Further, pharmaceutical guidelines limit the presence of particles (e.g. oil particles) permitted within a drug. Accordingly, for some applications, it would be desirable to have a means to feed a gasket into a prefilled syringe without flowable lubricant.

Applicant is currently developing a gasket design and methods of making the gasket, which, being currently internally developed, are not yet published and are not yet part of the state of the art. Applicant has found that this gasket has demonstrated improved performance when used in a syringe. However, Applicant's own experience testing this gasket design revealed that it has a greater propensity to deform radially when being fed into a syringe via a standard feeding tube through methods described above. Applicant has therefore determined that for this unique gasket configuration, there is a need for methods and related equipment that reliably facilitate a smooth feeding process without deforming the shape of the gasket and preferably without any or substantially without any oil for lubrication between the gasket and the feeding tube.

### SUMMARY OF THE INVENTION

Accordingly, in one optional aspect, a tube for retaining and feeding a syringe gasket into a medical barrel is provided. The tube includes an elongate hollow tubular body comprising an internal lumen, the lumen having an inner wall which includes a contact surface configured to contact an outer wall of a gasket. The contact surface has a surface area which is less than that of the inner wall.

In another aspect, the tube includes an elongate hollow tubular body including an internal lumen, the lumen having an inner wall which includes a contact surface configured to contact an outer wall of a gasket. The contact surface includes two or more guide rails extending axially along the lumen.

Optionally, in any embodiment, the contact surface includes a non-stick coating deposited thereon.

In one optional aspect, the disclosed solution reduces risks to the patient as there is no more introduction of silicone oil or other flowable lubricant particles into the drug preparation. Another advantage is that feeding of the gaskets no longer relies on even distribution of lubricants along glide surface areas.

Optionally, the disclosed concept is directed to systems for feeding a syringe gasket into a medical barrel. In one optional aspect, the system includes a gasket configured for insertion into a medical barrel. The gasket has a main body made of an elastic material, the gasket having a distal nose cone configured to face a product stored within the medical barrel when inserted into the medical barrel and a circumferential surface portion extending proximally from the nose cone. At least a portion of the circumferential surface portion is configured to contact an interior sidewall of the medical barrel in compressive engagement. The system further includes a tube for retaining the gasket and then feeding the gasket into the medical barrel. The tube includes an elongate hollow tubular body having an internal lumen. The lumen has an inner wall which consists of a contact surface configured to contact a portion of the circumferential surface portion of the gasket and a non-contact surface occupying the remainder of the inner wall. The gasket is disposed within the tube such that, along a selected plane perpendicular to a central axis of the tube, a first part of the circumferential surface portion of the gasket contacts and is in compressive engagement with the contact surface while a second part of the circumferential surface portion of the gasket either is not in contact with the non-contact portion, or is in contact with the non-contact portion but with less compression against the non-contact portion than exists between the first part of the circumferential surface portion of the gasket and the contact surface.

The disclosed concept is also directed to methods for feeding a syringe gasket into a medical barrel. Optionally, the methods employ use of components of the system described in the paragraph immediately above. In an optional aspect, the method includes placing the gasket into the tube such that, along a selected plane perpendicular to a central axis of the tube, a first part of the circumferential surface portion of the gasket contacts and is in compressive engagement with the contact surface while a second part of the circumferential surface portion of the gasket either is not in contact with the non-contact portion, or is in contact with the non-contact portion but with less compression against the non-contact portion than exists between the first part of the circumferential surface portion of the gasket and the contact surface. The tube is aligned with a proximal open end of the medical barrel. The gasket is slid through the tube and then transferred from the tube into the medical barrel.

Optionally, in any embodiment, the contact surface includes two or more guide rails extending axially along the lumen. Optionally, the guiderails are outwardly rounded along a cross-sectional plane perpendicular to the central axis of the tube. As an alternative option, the guiderails are inwardly rounded along the cross-sectional plane. Optionally, the tube comprising two to eight guiderails. Optionally, there are an even number of guiderails, wherein each guiderail opposes another guiderail located 180 degrees from it around the lumen.

Optionally, in any embodiment, the contact surface has a surface area which is less than that of the inner wall, optionally wherein the surface area is less than 75%, optionally less than 65%, optionally less than 50%, optionally less than 40%, optionally less than 30% of that of the inner wall, optionally from 15% to 50%, or optionally from 25% to 50%, of that of the inner wall.

Optionally, in any embodiment, the contact surface includes a surface coating having low surface energy. Optionally, the surface coating includes a sol-gel coating. Optionally, the surface coating includes an inorganic-organic hydride polymer, e.g., an ORMOCER® coating. Optionally, the surface coating includes a fluorinated polymer, optionally a member selected from the group consisting of: fluorinated ethylene propylene (FEP), ethylene tetrafluoroethylene (ETFE), polytetrafluoroethylene (PTFE), polychlorotrifluorocthylene (PCTFE) and perfluoroalkoxy (PFA).

Optionally, in any embodiment, the tube is composed of or comprises polyoxymethylene homopolymer, polytetrafluoroethylene or a melt blend of the two.

Optionally, in any embodiment, the tube is composed of or comprises polyoxymethylene homopolymer with polytetrafluoroethylene fibers uniformly blended in the polyoxymethylene homopolymer.

Optionally, in any embodiment, the circumferential surface portion of the gasket has one or more ribs that protrude radially outward and are configured to engage and provide a seal between the gasket and a medical barrel into which the gasket is disposed. At least one of the one or more ribs includes a channel or channels running along at least a portion of the circumference of the at least one of the one or more ribs. Optionally, the one or more ribs comprises a distal rib adjacent to the nose cone, the channel or channels being provided on the distal rib. Optionally, the channel or channels are laser cut. Optionally, the channel or channels are non-continuous.

Optionally, in any embodiment of the gasket, a film resides on at least a part of the circumferential outer portion of the gasket.

Optionally, in any embodiment, the gasket includes the main body having an internal cavity, the cavity being defined by an inner surface portion of the gasket and being open-ended at one end. A film resides on at least a part of the circumferential surface portion of the gasket. A plurality of non-continuous channels in or through the film are approximately parallel to the other non-continuous channels. Each non-continuous channel of the plurality of non-continuous channels extend around the circumferential outer surface of the gasket and have a non-channel portion interrupting the non-continuous channel. The non-channel portion of each non-continuous channel is positioned along the circumferential outer surface portion of the gasket such that it is not aligned with the non-channel portion of the immediately adjacent non-continuous channel. Optionally, the plurality of non-continuous channels are provided on a rib of the gasket, preferably a distal rib adjacent to the nose cone.

Optionally, in any embodiment of systems or methods disclosed herein, there is no flowable lubricant in the tube.

### BRIEF DESCRIPTION OF SEVERAL VIEWS OF THE DRAWINGS

The background of the invention and the invention itself will be described in conjunction with the following drawings in which like reference numerals designate like elements and wherein:

FIG. 1 is a sectional view of a syringe comprising a syringe barrel assembled with a gasket attached to a plunger rod.

FIG. 2 is a fragmentary detail view of the syringe of FIG. 1.

FIG. 3A is a schematic sectional view taken along section lines 3A-3A of FIG. 2.

FIG. 3B is a fragmentary detail view of the structure of FIG. 3A, showing one embodiment of each of the non-continuous channels extending around the circumferential outer surface of the gasket core and approximately parallel to first non-continuous channel and lips on the respective sides of the channel.

FIG. 3C is a top view of the gasket showing the approximate geometric distribution of the non-continuous channels extending around the circumferential outer surface of the gasket core.

FIG. 3D is a top view of the gasket showing the approximate geometric distribution of three non-continuous channels extending around the circumferential outer surface of the gasket core, which are axially spaced from each other, each of which including a respective non-channel portion.

FIG. 4A is an assembly of a gasket and a mandrel inserted into the internal cavity of the gasket and schematic depiction of a laser beam being applied obliquely to an outer portion of a film residing on a circumferential outer surface portion of the gasket while the mandrel and gasket are being rotated along the mandrel's longitudinal axis to produce a non-continuous channel in the film residing on a circumferential outer portion of the gasket.

FIG. 4B is a schematic sectional view of one embodiment of the gasket taken along section lines 4B-4B of FIG. 4A, showing the mandrel secured in the gasket's internal cavity and non-continuous channels in the outer surface of the film residing on a circumferential outer surface portion of the gasket.

FIG. 5A is a gasket with a film having a first non-continuous channel in the film.

FIG. 5B is a fragmentary detail view of the gasket of FIG. 5A, showing an embodiment of a first non-continuous channel in the surface of the film with lips on the respective sides of the first non-continuous channel and the various dimensions of the first non-continuous channel.

FIG. 6 is a schematic illustration, shown for background purposes, of a conventional system for feeding a gasket into a prefilled syringe.

FIG. 7 is a cross sectional view of a prior art feeding tube.

FIG. 8 is a cross sectional view of a feeding tube according to a first optional embodiment of the disclosed concept.

FIG. 9 is a cross sectional view of a feeding tube according to a second optional embodiment of the disclosed concept.

FIG. 10 is a cross sectional view of a feeding tube according to a third optional embodiment of the disclosed concept.

FIG. 11 is a cross sectional view of a feeding tube according to a fourth optional embodiment of the disclosed concept.

### DETAILED DESCRIPTION OF PREFERRED EMBODIMENTS OF THE INVENTION

The disclosed concept will now be described more fully with reference to the accompanying drawings, in which several embodiments are shown. This invention may, however, be embodied in many different forms and should not be construed as limited to the embodiments set forth here. Rather, these embodiments are examples of the invention, which has the full scope indicated by the language of the claims. Like numbers refer to like elements throughout. Unless indicated otherwise, the features characterizing the embodiments and aspects described in the following disclosure may be combined with each other, and the resulting combinations are also embodiments of the disclosed concept.

### Definitions

As used in this disclosure, the term “syringe” is a device comprising a medical barrel and a gasket disposed within the medical barrel to dispense liquid contents within the medical barrel or to draw liquid into the medical barrel. A syringe is broadly defined to include cartridges. injection “pens,” and other types of barrels or reservoirs adapted to be assembled with one or more other components to provide a functional syringe. “Syringe” is also broadly defined to include related articles such as auto-injectors, which provide a mechanism for dispensing the contents. Optionally, “syringe” may include prefilled syringes. A “syringe” as used herein may also have applications in diagnostics, e.g., a sampling device comprising a medical barrel prefilled with a diagnostic agent (e.g., contrast dye) or the like. Though the disclosure is not necessarily limited to syringes of a particular volume, syringes are contemplated in which the lumen has a void volume of, for example, from 0.5 to 50 mL, optionally from 1 to 10 mL, optionally from 0.5 to 5 mL, optionally from 1 to 3 mL.

As used herein, the term “gasket” in the context of the present disclosure is a shaped piece or ring made of an elastomeric material that can be used to mechanically seal the space between two opposing inner surfaces of a syringe barrel. The gasket is also referred to as a plunger, A gasket is preferably cylindrical in shape with a short axis. The gasket has a circumferential surface portion to be kept in substantially gas-tight and liquid-tight contact with an inner peripheral surface of the syringe barrel. A gasket of the present disclosure is a gasket comprising a main body made of an elastic material and a film residing on at least a circumferential surface of the main body, the gasket having a circumferential surface portion and an internal cavity (IC) in its center, the cavity being defined by the inner surface of the gasket and being open at one end.

The “elastic material” may be rubber or an elastomer. Particularly preferred types of rubber include butyl rubbers, chlorinated butyl rubbers and brominated butyl rubbers. Other types of elastic material may include thermosetting rubbers and dynamically cross-linkable thermoplastic elastomers having crosslinking sites are which make them heat-resistant. These polymer components of such elastomers include ethylene-propylene-diene rubbers and butadiene rubbers.

As used herein, the term “film” is a material residing on at least a circumferential outer surface portion of the main body of the gasket. Preferably, it coats or resides on substantially all of the outer surfaces of the gasket. The film may have an optional thickness of under about 100 micrometers (μm or microns), optionally from about 10-30 microns, about 15-35 microns, or about 20-50 microns. Most preferably, the film is about 20 microns in thickness. A variety of different materials may be employed for the film, such as, for example, an inert fluoropolymer, including, fluorinated ethylene (FEP), propylene ethylene tetrafluoroethylene (ETFE), polytetrafluoroethylene (PTFE), ethylene perfluoroethylenepropylene (EFEP), ethylene chlorotrifluoroethylene (ECTFE), Polychlorotrifluoroethene (PCTFE), perfluoroalkoxy (PFA), among other coatings. Preferably, the film is an ultrahigh molecular weight polyethylene film (UHMWPE) or a fluoropolymer film. Fluoropolymer films such as polytetrafluoroethylene (PTFE) are preferred because of their excellent slidability and chemical stability. The type of the film to be provided on the surface of the main body of the gasket is not particularly limited, as long as the film is capable of preventing migration of substances from the crosslinked rubber (main body) and has a slidability, i.e., a smaller friction coefficient, as compared to the main body of the gasket.

Optionally, the film may comprise CPT fluoropolymer. CPT is a modified perfluoroalkoxy (PFA) that generally comprises the addition of PCTFE side chains to a PFA main chain during polymerization.

As used herein, the term “channel” refers to a cut in the film residing on the surface of the gasket, preferably by a laser cut. The term channel may be used interchangeably with the term “cut.” In the present disclosure, the term “cut” may also refer to the process of using one or more laser beams to create a nick or separation of the film residing on at least a circumferential outer surface portion of a gasket. In some embodiments, the channel is cut in the surface portion of the film. In more preferred embodiments, the channel extends through the film into the outer surface of the gasket. One or more such channels can be produced, each encircling a part of the gasket. Each channel has a non-channel portion where the channel/cut is not formed. For example, the channel can encircle 350 degrees of the gasket and the non-channel portion can encircle the remaining 10 degrees of a 360 degree circle on the gasket. When more than one channel is present, they are preferably axially spaced from one another. The non-channel portions of the more than one channels are not aligned on the gasket. For example, the non-channel portion can be disposed on one side, and a second non-channel portion can be disposed on another side of the gasket. Each channel has two lips. The term “lip” refers to the structure created due to the pile-up of film material along either side of the channel that is created by the laser beam cut. Each lip is a raised micro projection positioned to seal against the barrel's inner surface. Thus, each channel has two lips comprising two scaling micro projections or peaks. In the present disclosure, the terms “lip”. “peak” and “micro projection” are interchangeable.

The laser cut and the resulting channels are characterized by various dimensions, including laser-cut depth, radial depth, peak width, axial width, and peak height. The “laser-cut depth” is measured from the surface of the uncut gasket film down to the lowest point in the trough of the channel. The laser-cut depth for the one or more channels is independently selected from the following ranges: 30-60 microns, 40-50 microns, 50-60 microns, 40-45 microns. 45-50 microns, 50-55 microns and 55-60 microns. The “radial depth” is measured from the uncut outer surface of the gasket up to the lowest trough in the channel. The radial depth for the one or more channels that may be independently selected from the following ranges: 0 to 100 microns, 5 to 50 microns, 10 to 30 microns, and 15 to 25 microns. The “peak width” is the distance between two peaks of two lips on either side of a channel. Peak width is measured from the top of the peaks. The peak width may be one of the following ranges: 200-1,000 micron, 275-550 microns, 300-400 microns, and 450-500 microns.

The circumferential non-continuous channel of the present disclosure has axially opposed “first and second side walls” and a “floor.” The floor of the channel may be either a film surface or, more preferably a gasket surface, depending on the thickness of the film and the depth of the cut. The “axial width” is measured from the first side wall to the second side wall of the channel across the breadth of the channel floor. In other words, the “axial width” is measured from one end of a channel to the other end of the channel across its breadth at the baseline level, i.e., at the laser uncut outer surface level of the film or gasket. The one or more channel independently has an axial width between the side walls of one of the following ranges: 1 to 100 microns, 5 to 50 microns, 10 to 30 microns, and 15 to 25 microns.

The “peak height” is measured from the surface of the uncut gasket film up to the highest peak of the lip created by the laser beam along the central axis of the peak, i.e., perpendicular to the surface of the film. The peak height of the lip on one or more of the channels is independently selected from one of the following ranges: 10-100 microns, 15-60 microns, 20-50 microns, and 30-40 microns.

### Feeding Tubes According to the Disclosed Concept

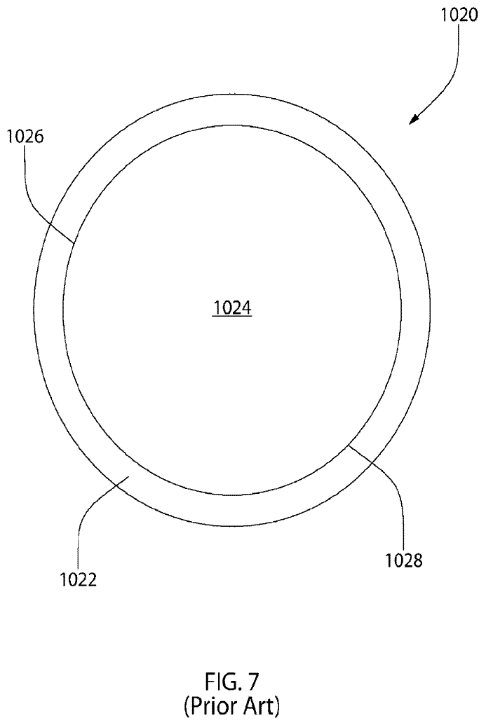

As noted in the Background section above, the novel syringe gasket which Applicant has developed internally, while an improvement over the state of the art, has a greater propensity than convention gaskets to deform radially when being fed into a syringe through a standard feeding tube (e.g., the prior art feeding tube described in the Background section and shown in FIG. 7). In response to this technical challenge, and as described in the present disclosure. Applicant has determined that novel and alternative designs for feeding tubes are needed to overcome the aforementioned problem. It is a further object of the disclosed concept that minimal or no flowable lubricant (e.g., silicone oil) should be needed to feed the gasket through a feeding tube and into a syringe barrel.

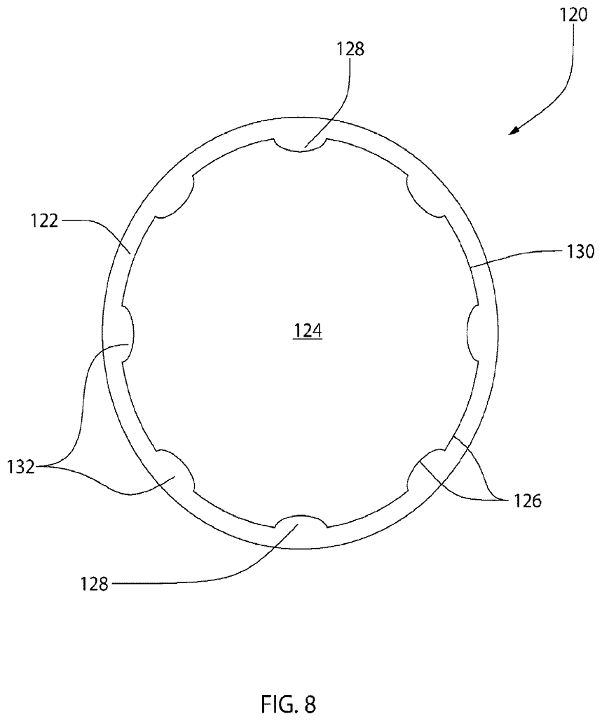

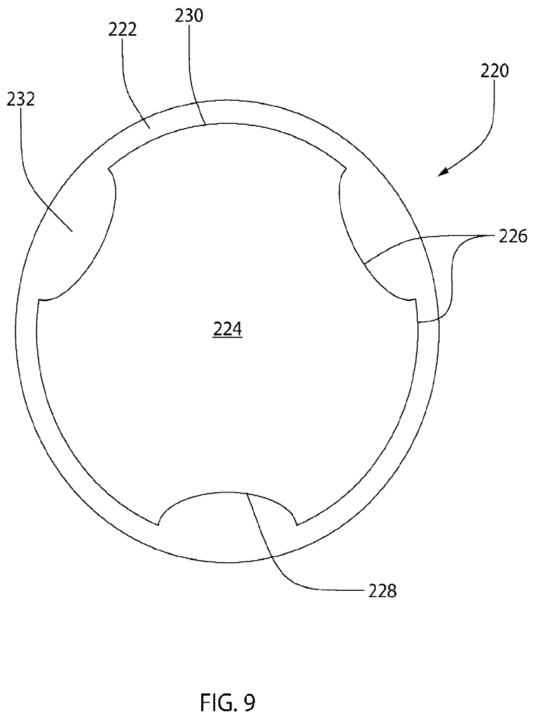

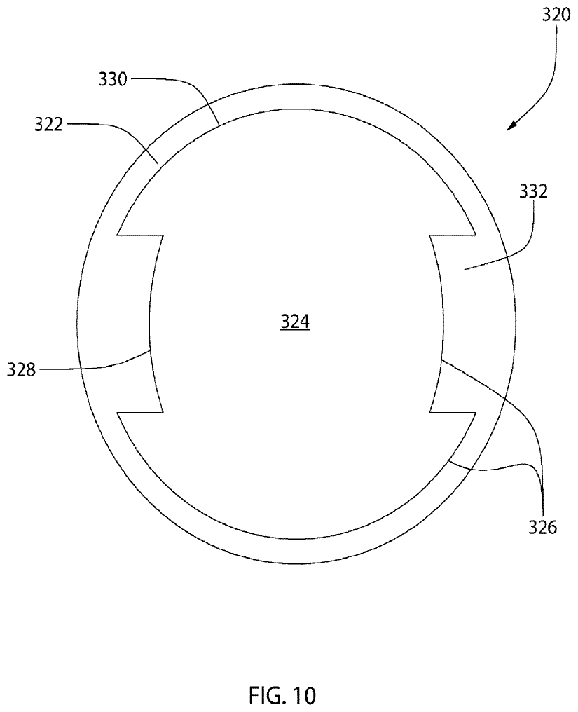

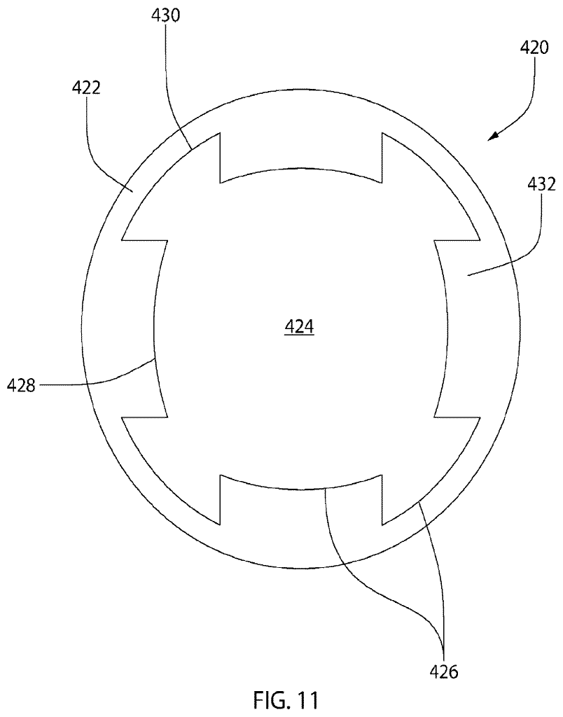

Accordingly, referring now to FIGS. 8 to 11, optional embodiments of feeding tubes 120, 220, 320, 420 according to the disclosed concept are shown. Each of these views is a cross section along a plane perpendicular to the central axis of a given feeding tube 120, 220, 320, 420, illustrating the internal geometry of the feeding tube 120, 220, 320, 420. Each feeding tube 120, 220, 320, 420 includes a hollow tubular body 122, 222, 322, 422 having an inner wall 126, 226, 326, 426 defining a lumen 124, 224, 324, 424, The lumen 124, 224, 324, 424 is configured to have a syringe plunger, i.e., gasket, placed therein, prior to feeding the gasket into a syringe barrel. The inner wall 126, 226, 326, 426 includes a contact surface 128, 228, 328, 428 configured to contact and optionally provide compressive engagement with the outer wall of a gasket disposed within the lumen 124, 224, 324, 424.

Optionally, in any embodiment, the feeding tube 120, 220, 320, 420 is constructed from or comprises a material that may be machined or molded to precise measurements and tolerances. The material may be selected from a wide range of materials. Such materials may include, e.g., metals or plastics that provide for a low friction contact surface 128, 228, 328, 428. For example, if a plastic material is used, the plastic may optionally be selected from the group consisting of: polystyrene, polycarbonate, polypropylene, polyethylene, polytetrafluoroethylene, ethylene tetrafluoroethylene, and any combination of the foregoing.

In preferred embodiments, the feeding tube 120, 220, 320, 420 is constructed from or comprises polyoxymethylene homopolymer (also known as acetal homopolymer) or comprises polyoxymethylene copolymer, or the like. The polyoxymethylene homopolymer may be formulated as neat homopolymer or may be formulated in a blend with polytetrafluoroethylene (PTFE). The blend may be formulated by melting both the polyoxymethylene homopolymer and the PTFE and blending the two polymers. Or, the polyoxymethylene homopolymer may be melted and PTFE fibers may be blended into the homopolymer. This material can be used in moving parts providing low friction and long wear life.

Unlike the inner wall 1026 and contact surface 1028 of the prior art feeding tube 1020, the inner wall 126, 226, 326, 426 and contact surface 128, 228, 328, 428 of the feeding tubes 120. 220, 320, 420 according to the disclosed concept are not one and the same; they are not coextensive. Rather, the contact surface 128, 228, 328, 428 has a surface area which is less than that of the inner wall 126, 226, 326, 426. As such, the inner wall 126, 226, 326, 426 consists of only the contact surface 128, 228, 328, 428 and a non-contact surface 130, 230, 330, 430 (which occupies the remainder of the inner wall 126, 226, 326, 426). Mathematically, this may be expressed as A_{IW}=A_{CS}+A_{NC} (and its corollaries A_{IW}−A_{CS}=A_{NC} and A_{IW}−A_{NC}=A_{CS}), where A_{IW}=area of the inner wall, A_{CS}=area of the contact surface and A_{NC}=area of the non-contact surface.

The non-contact surface 130, 230, 330, 430, unlike the contact surface 128, 228, 328, 428 is configured not to contact or at least to provide lesser contact force against (i.e., reduced compressive engagement with) the outer wall of a gasket disposed within the lumen 124, 224, 324, 424. With a contact surface 128, 228, 328, 428 having a smaller surface area than the total inner wall 126, 226, 326. 426, feeding tubes 120, 220, 320, 420 according to the disclosed concept provide reduced frictional resistance to a gasket along the gasket's travel path compared to the prior art. Optionally, in any embodiment, the surface area of the contact surface 128, 228, 328, 428 is less than 75%, optionally less than 65%, optionally less than 50%, optionally less than 40%, optionally less than 30%, optionally from 15% to 50%, or optionally from 25% to 50%, of the total inner wall 126, 226, 326, 426 surface area.

Accordingly, optionally, in any embodiment, the inner wall 126, 226, 326, 426 is not perfectly round.

The precise geometric configuration of the contact surface 128, 228, 328, 428 may vary, depending on the application, relative size and materials of the gaskets used and other factors. Optionally, the contact surface 128, 228, 328, 428 may comprise two or more guide rails 132, 232, 332, 432 protruding radially inward from the non-contact surface 130, 230, 330, 430 and extending axially along the length of the lumen 124, 224, 324, 424. The guide rails 132, 232, 332, 432 are preferably evenly spaced from one another in order to stabilize the gasket as it is advanced down the feeding tube 120, 220, 320, 420. The guide rails 132, 232, 332, 432 are configured to provide a reduced area sliding surface for a gasket that is being advanced down the lumen 124, 224, 324, 424. Optionally, the guide rails 132, 232, 332, 432 provide inwardly or outwardly rounded contact surfaces 128, 228, 328, 428. For example, contact surfaces 128, 228 are convex while contact surfaces 328, 428 are concave, The number of guide rails 132, 232, 332, 432 may vary as well, as shown in the different exemplary embodiments.

To further minimize friction, the contact surfaces 128, 228, 328, 428 should have low surface roughness and optionally comprise a surface coating having low surface energy disposed thereon. This may be done using a wide range of materials. For example, a thin surface coating with low surface energy could be added using “baked on” pharmaceutical grade coatings like selected Sol-Gel coatings (ORMOCER® coatings from Fraunhofer Institute) or other suitable permanently bound non-stick coatings.

ORMOCER@ coatings, which are organic modified ceramic polymers, are understood as being inorganic-organic hydride polymers. They are silicone polymers which are known as coating material for metals, glass, stone, etc. The preparation and composition of the inorganic-organic hybrid polymers are described, for example, in DE 43 03 570 C and EP0610831B1, both of which are incorporated by reference herein in their entireties.

Optionally, an ORMOCER@ coating is a hybrid organic-inorganic polymeric coating comprising, consisting essentially of, or consisting of:
(i) a hydrolytic condensate, prepared from a silane of the formula RₘSiX₄₋ₘ having the following meanings:
   R=crosslinkable organic radical
   X=hydrolyzable and condensable group
   m=1 or 2 or 3 (with 1 being preferred)
   with a metal compound.
(ii) a prepolymer that is crosslinkable with the radicals R of the silane.
(iii) one or more (especially one or two) optional non-crosslinkable organofunctional silane(s) and
(iv) an optional low-volatility oxide.

Alternatively, the inner wall may have a coating of or otherwise comprise a polymer having low surface energy. This may include a fluorinated polymer, optionally a member selected from the group consisting of: fluorinated ethylene propylene (FEP), ethylene tetrafluoroethylene (ETFE), polytetrafluoroethylene (PTFE), polychlorotrifluoroethylene (PCTFE) and perfluoroalkoxy (PFA).

The non-stick coatings preferably withstand typical sterilization methods, especially autoclaving, gas treatment and/or radiation. The coatings should also be suitable for pharmaceutical use, biocompatible and free of cytotoxins. The coatings should also be of a nature and applied in such a way as to prevent delamination. With the reduced contact surface area and low surface energy coating, it is contemplated that the need for silicone oil or other flowable lubricants would be obviated. In this regard, the disclosed concept provides an oil-free solution.

Syringes and syringe plungers (gaskets) are often configured according to ISO Standards, It is therefore possible to manufacture apparatus, including feeding tubes, to accommodate the various standard sizes of medical barrels and gaskets. The feeding tubes 120, 220, 320, 420 described herein would typically be part of a larger machine that precisely locates one or multiple syringes below a series of one or more gasket insertion apparatuses and then delivers and inserts the gaskets within the pre-filled syringes. The basic function of this system is the insertion of gaskets (also called plungers, pistons or stoppers) into filled syringes. The syringes are filled with a liquid medium, e.g., liquid drug product. The main functional elements for this insertion are gasket feeding tubes and gasket rams. Each functional element is moved e.g. by mechanical cams, servo motors or robots relative to each other according to a specific timing. A gasket insertion system includes one plunger feeding tube and gasket ram for placing and sealing one syringe at once or more sets for sealing two or more syringes at the same time.

In the case of a system for placing and scaling a single gasket into a single syringe, the system operates as follows. The gasket is placed concentrically above the feeding tube. By a vertical movement of a gasket ram, the gasket is inserted into the feeding tube. The gasket is slightly compressed and thus retained by a slight interference fit within the feeding tube. The gasket ram then advances the gasket down the feeding tube and into the medical barrel to a predetermined depth. The feeding tube then begins to move upward, followed by upward movement of the gasket ram, so as to release the gasket above the fill level within the medical barrel. The gasket ram and feeding tube move to their respective home positions to restart the process with a new gasket and medical barrel. As an alternative, using more simplified machinery. a gasket insertion system may use a vacuum to assist the insertion of the gasket, as described above in the Background section.

### Optional Gaskets for Use in Feeding Tubes According to the Disclosed Concept

The gasket that is to be used in conjunction with the feeding tube 120, 220, 320, 420 is optionally any gasket suitable for feeding via a feeding tube into a syringe, such as conventional gaskets in the field.

Optionally, the gasket that is to be used in conjunction with the feeding tube 120, 220, 320, 420 is a gasket having one or more channels along the circumferential surface portion of at least one gasket rib, optionally a distal rib, adjacent a gasket nose cone. Preferably, such channel(s) is non-continuous, i.e., it does not extend an entire 360 degrees around the circumference of the rib.

Optionally, in one aspect of gaskets having channels along the gasket's side and which may be used in connection with feeding tubes according to the disclosed concept, the gasket is that noted above which Applicant developed internally and which gave rise to Applicant's recognition for the need for innovation in the configuration of feeding tubes. As a summary, this gasket has a circumferential surface portion along a gasket sidewall and a film residing on at least a part of a circumferential outer surface portion of the gasket. The film is optionally a fluoropolymer film to facilitate slidability of the gasket, both during feeding and in use for dispensing syringe contents. The gasket includes an internal cavity in its center. The gasket is characterized by a plurality of non-continuous channels in or through the film, each non-continuous channel being approximately parallel to the other non-continuous channels. Each non-continuous channel of the plurality of non-continuous channels extend around the circumferential outer surface of the gasket and have a non-channel portion interrupting the non-continuous channel. The non-channel portion of each non-continuous channel is positioned along the circumferential outer surface portion of the gasket such that it is not aligned with the non-channel portion of the immediately adjacent one or more non-continuous channels.

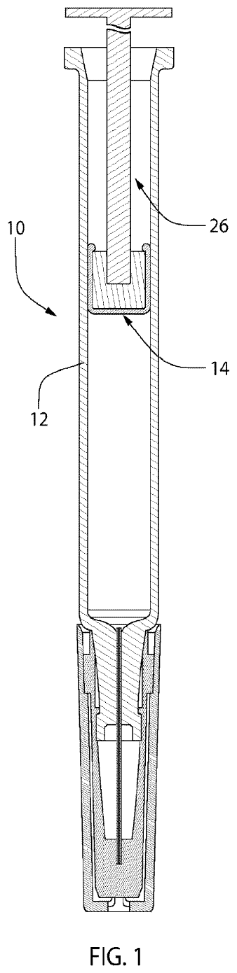

Further details regarding optional gaskets that may be used in conjunction with feeding tubes 120, 220, 320, 420 according to the disclosed concept are now provided with reference to FIGS. 1-5B.

FIG. 1 shows a syringe 10 of the present disclosure which includes a hollow cylindrical syringe barrel 12, a plunger rod 26 combined with the syringe barrel 12 and reciprocally movable in the syringe barrel 12, and a gasket 14 attached to a distal end of the plunger rod 26. The syringe barrel 12 may be similar to, substantially similar to or identical to medical barrel 1012, described above.

As used herein, the terms “distal” and “proximal” generally refer to a spatial or positional relationship relative to a given reference point, wherein “proximal” is a location at or comparatively closer to that reference point and “distal” is a location further from that reference point. As applied herein to the plunger rod 26, for example, the relevant reference point is the bottom end of the plunger rod 26, the distal end, which is attached to the gasket 14. As applied herein to the syringe barrel 12, for example, the relevant reference point is the bottom end of the barrel 12, the distal end, which is attached to a delivery conduit or hypodermic needle.

The syringe 10 is of generally conventional construction and materials, preferably plastic, and includes a hollow barrel 12 having a central longitudinal axis. The barrel has an inner surface 14 and is configured to hold an injectable liquid therein. A hypodermic needle or delivery conduit is located at the distal end of the barrel and is in fluid communication therewith. The plunger rod 26 is also of generally conventional construction and materials. A gasket 14 of the disclosure (shown schematically in FIG. 1) is attached to the distal end of the plunger rod 26.

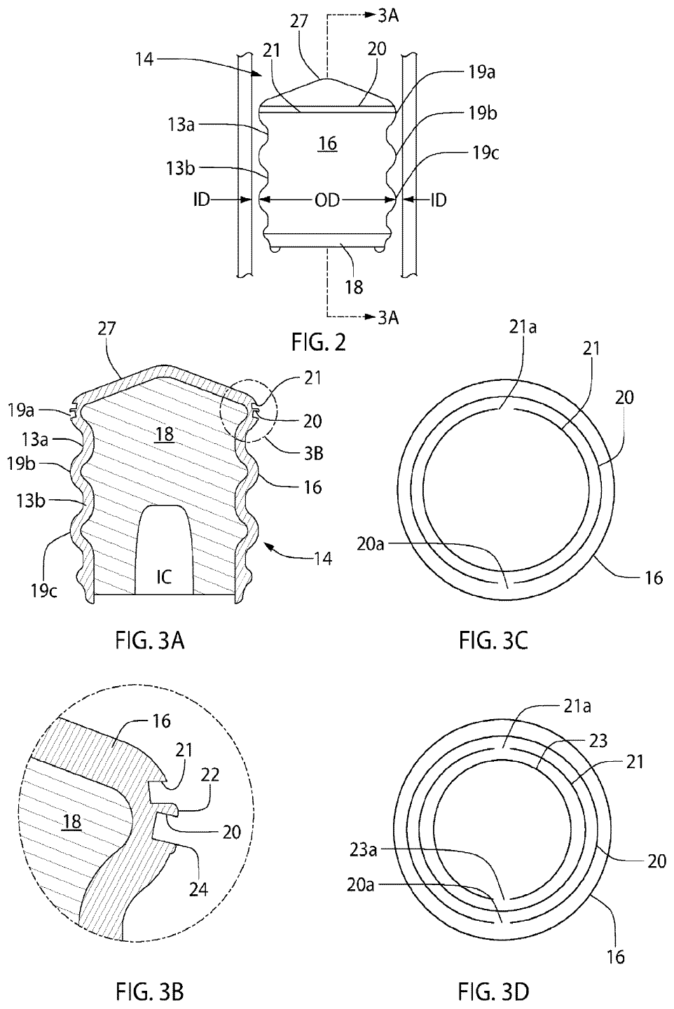

FIG. 2 shows a fragmentary detail view of the syringe of FIG. 1, showing the inner diameter (ID) of the barrel 12 and the outer diameter (OD) of the gasket 14 that are matched to be within the predetermined tolerance between them, and also showing a film 16 covering the outer surface of a gasket core 18, The gasket 14 preferably includes a nose cone 27 at a distal end thereof and one or more ribs 19a, 19b, 19c that protrude radially outward (relative to the gasket's central axis) from a sidewall of the gasket 14 to engage and provide a liquid tight and/or gas tight seal between the gasket 14 and the barrel 12. In embodiments where there are at least two ribs, a valley 13a, 13b separates each such rib. Such configurations provide surface contact and compression via the ribs (e.g., 19a, 19b, 19c) that are necessary for a good seal to protect the quality of a drug product contained within the syringe 10. At the same time, the valleys (e.g., 13a, 13b), which have a lesser outer diameter than the ribs (e.g., 19a, 19b, 19c) provide areas of comparatively reduced compression or no contact or compression with the barrel 12. Thus, the ribs and valleys together balance the competing goals of providing a good seal without excessive compression/friction to allow for a reasonably low and consistent plunger force when the plunger rod 26 is pressed distally down the barrel 12 to dispense contents of the syringe 10.

In one optional aspect of the disclosed concept, at least one non-continuous channel extends around an outer surface of the gasket. For example, a first non-continuous channel 20 extends around the circumferential outer surface of the gasket core 18 and a second non-continuous channel 21 extends around the circumferential outer surface of the gasket core 18. The second non-continuous channel 21 is optionally approximately parallel (i.e., in respective parallel planes) to first non-continuous channel 20. It is preferred that the channel or channels (e.g., 20 and 21) are provided on a rib (e.g., 19a, 19b and/or 19c) of the gasket 14, more preferably on the distal rib 19a, or solely on the distal rib 19a.

FIG. 3A shows a schematic sectional view taken along section lines 3A-3A of FIG. 2. showing the gasket core 18 within internal cavity (IC), film 16, and first non-continuous channel 20 in the surface of the film (in some embodiments channel 20 extends through the film into the outer surface of the gasket) and second non-continuous channel 21 in the surface of the film (in some embodiments channel 21 extends through the film into the outer surface of the gasket) and approximately parallel to first non-continuous channel 20. As shown in this optional embodiment, the non-continuous channels 20, 21 are located on the distal rib 19a.

FIG. 3B is a fragmentary detail view of the structure of FIG. 3A, showing one embodiment of each of the first non-continuous channel 20 and the second non-continuous channel 21 extending around the circumferential outer surface of the gasket core 18. As shown, the second non-continuous channel 21 is approximately parallel to first non-continuous channel 20 and lips 22 and 24 on the respective sides of the channel 20.

FIG. 3C shows a top view of the gasket 14 and the approximate geometric distribution of the first non-continuous channel 20 extending around the circumferential outer surface of the gasket core 18 and the approximate geometric distribution of the second non-continuous channel 21 extending around the circumferential outer surface of the gasket core 18. The top view is intended to be illustrative of the position of the non-channel portion for each non-continuous channel of the plurality of non-continuous channels. Each of the non-continuous layers thus includes a non-channel portion (21a, 20a) associated with the respective non-continuous layers (21, 20). The non-channel portions (21a, 20a) are not aligned with each other on the circumferential outer surface portion of the gasket. Although FIG. 3C illustrates the non-channel portion of the first non-continuous channel being positioned 180 degrees from the non-channel portion of the adjacent non-continuous channel around the circumference of the gasket 14, the non-channel portions may be located at alternative positions around the circumference of the gasket 14 as long as the adjacent non-channel portions are not aligned. For example, the non-channel portion of the first non-continuous channel may be positioned in a range of 90 to 270 degrees from the adjacent non-channel portion around the circumference of the gasket, with the adjacent non-channel portions not aligned. In a preferred embodiment, the non-channel portion of the first non-continuous channel may be positioned approximately 180 degrees from the adjacent non-channel portion around the circumference of the gasket.

Optionally, in another embodiment, each non-continuous channel may include a plurality of non-channel portions arranged around the circumference such that the non-continuous channel may be a “dashed line” of channel and non-channel portions. An adjacent non-continuous channel may also be a “dashed line” channel of channel and non-channel portions. The channel and non-channel portions of adjacent non-continuous channels may be positioned around the circumference of the gasket as long as the adjacent non-channel portions are not aligned.

FIG. 3D shows a top view of an optional embodiment of a gasket and the approximate geometric distribution of three non-continuous channels 20, 21, 23 extending around the circumferential outer surface of the gasket core. The top view is intended to be illustrative of the position of the non-channel portion for each non-continuous channel 20, 21, 23 of the plurality of non-continuous channels, Each of the channels 20, 21, 23 extends around the circumferential surface of the gasket and each having a portion that does not include a channel. The three non-continuous channels 20, 21, 23 are axially spaced from each other and are arranged such that non-continuous channel 20 is proximal to the top of the gasket relative to the other two non-continuous channels 21 and 23, non-continuous channel 21 is adjacent to non-continuous channel 20 and distal to the top of the gasket relative to non-continuous channel 20, and non-continuous channel 23 is adjacent to non-continuous channel 21 and distal to the top of the gasket relative to non-continuous channel 21. Each of the non-continuous channels 20, 21, 23 includes a non-channel portion (20a, 21a, 23a) associated with the non-continuous channels 20, 21, 23. The non-channel portions (20a, 21a, 23a) are positioned such that they are not aligned with the non-channel portion of the adjacent non-continuous channels along the circumferential outer surface portion of the gasket. In some embodiments, the non-channel portion of one non-continuous channel may be aligned with the non-channel portion of a non-adjacent non-continuous channel. This is exemplified with the relative location of non-channel portion 20a compared to that of non-channel portion 23a.

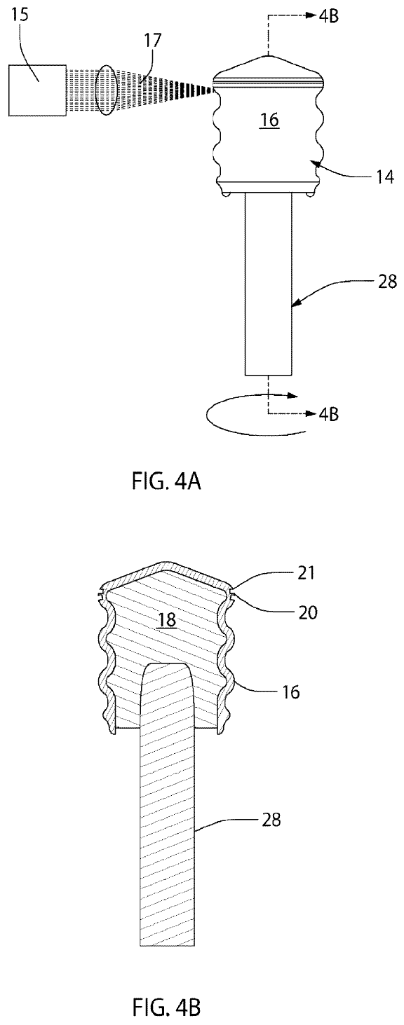

The channels may be formed by laser cutting. FIG. 4A shows a schematic illustration of a laser cutting process in one optional aspect of the present disclosure. As shown, a laser beam 17 is applied at a desired angle to the circumferential surface portion of the gasket 14 which is secured on a mandrel 28. The angle at which the laser beam 17 is applied can be 5 degrees to 90 degrees from the surface portion of the gasket 14. For the formation of channels in at least part of the circumferential surface portion of the gasket 14, a laser beam source 15 is fixed with respect to the circumferential surface portion of the gasket 14 with a film 16 residing on the outer surface of the gasket 14, and the laser beam 17 is applied to the circumferential surface portion while the gasket 14 secured to the mandrel 28 is rotated about the longitudinal axis thereof. Thus, the laser beam 17 can be applied at the predetermined incident angle α (e.g., 5° to 90°) to any angular position of the circumferential surface portion, whereby the channel is formed uniformly.

While the laser beam 17 is applied obliquely to the circumferential surface portion, the gasket may be rotated in a direction such that the circumferential surface portion is moved away from a laser beam application position at which the laser beam 17 is applied. Optionally, e.g., as shown in FIG. 4A, the gasket may be rotated clockwise.

FIG. 4B shows a schematic sectional view of the gasket 14 of 4A taken along section lines 4B-4B. As shown, film 16 resides on the gasket 14, with a first non-continuous channel 20 and a second non-continuous channel 21 in the outer surface of the film 16.

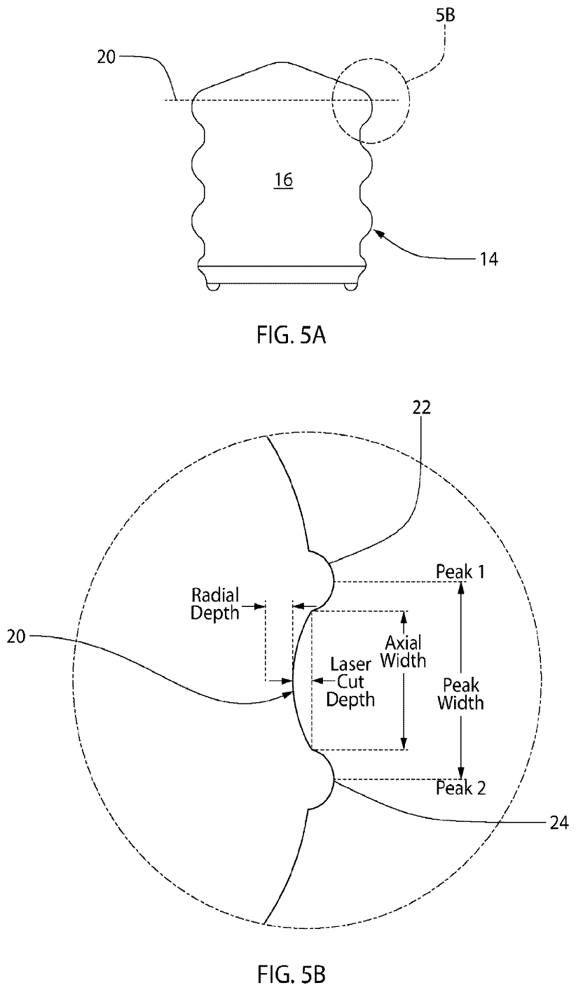

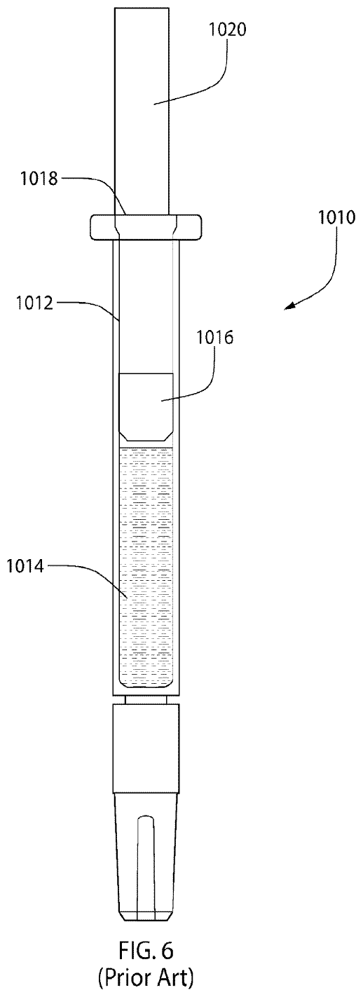

FIG. 5A shows a gasket 14 with a film 16 having first non-continuous channel 20 in the film 16. FIG. 5B shows a fragmentary detail view of the structure of FIG. 5A, showing an embodiment of a first non-continuous channel 20 in the surface of the film with lips 22 and 24 on the respective sides of the first non-continuous channel 20 and the various dimensions of the first non-continuous channel 20 and lips 22 and 24, including peak width, axial width, laser-cut depth and radial depth (these terms are defined above). In other, more preferred embodiments, first non-continuous channel 20 extends through the film 16 into the outer surface of the gasket 14.

By performing the laser cutting process described above, the channel is substantially uniformly formed in the film 16 and more preferably extending into the circumferential surface portion of the gasket and, at the same time, the outer edge portions 22 and 24 are formed, as shown in FIG. 5B.

### Optional Methods of Feeding Gaskets Into Medical Barrels According to the Disclosed Concept

In an optional aspect, a method of feeding a gasket into a medical barrel is provided. The method comprises:
providing a feeding tube 120, 220, 320, 420 according to any embodiment of the disclosed concept;
placing a gasket having an outer wall (circumferential surface portion) into the feeding tube 120, 220, 320, 420 within the lumen 124, 224, 324, 424 such that compression and/or interference fit between the outer wall against the contact surface 128, 228, 328, 428 retains the gasket in place within the lumen 124, 224. 324, 424;
aligning the feeding tube 120, 220, 320, 420 with a proximal open end of a medical barrel;
sliding the gasket through the feeding tube 120, 220, 320, 420 such that the outer wall of the gasket contacts only the contact surface without contacting the entirety of the inner wall 126, 226, 326, 426; and
transferring the gasket from the feeding tube 120, 220, 320, 420 into the medical barrel.

Optionally, use of the feeding tube 120, 220, 320, 420 as disclosed herein does not introduce any flowable lubricant particles into the syringe.

Optionally, in any embodiment, the gasket that is fed through the feeding tube is a gasket selected from any embodiments disclosed herein or conventional gaskets in the field.

Accordingly, the disclosed concept provides modified feeding tubes with inherently low friction. Use of two or more guide rails, as disclosed herein, defines the contact surface and axial travel path of the gasket, instead of having full surface contact with the tube.

### Exemplary Embodiments

The following exemplary embodiments further describe optional aspects of the presently disclosed technology and are part of this Detailed Description. These exemplary embodiments are set forth in a format substantially akin to claims, although they are not technically claims of the present application. The following exemplary embodiments refer to each other in dependent relationships as “embodiments” instead of “claims.”

1A. A tube for retaining and feeding a syringe gasket into a medical barrel, the tube comprising an elongate hollow tubular body comprising an internal lumen, the lumen having an inner wall which includes a contact surface configured to contact an outer wall of a gasket. wherein the contact surface has a surface area which is less than that of the inner wall, optionally wherein the surface area is less than 75%, optionally less than 65%, optionally less than 50%, optionally less than 40%, optionally less than 30% of that of the inner wall, optionally from 15% to 50%, or optionally from 25% to 50% of that of the inner wall.

1B. A tube for retaining and feeding a syringe gasket into a medical barrel, the tube comprising an elongate hollow tubular body comprising an internal lumen, the lumen having an inner wall which includes a contact surface configured to contact an outer wall of a gasket, the contact surface comprising two or more guide rails extending axially along the lumen.

1C. The tube of embodiment 1A or 1B, wherein the tube is composed of or comprises polymer or metal.

2C. The tube of embodiment 1C, wherein the tube is composed of or comprises a polymer selected from the group consisting of: polyoxymethylene homopolymer, polyoxymethylene copolymer polystyrene, polycarbonate, polypropylene, polyethylene, polytetrafluoroethylene, ethylene tetrafluoroethylene, and any combination of the foregoing.

3C. The tube of embodiment 2C, wherein the tube is composed of or comprises polyoxymethylene homopolymer.

4C. The tube embodiment 3C, wherein the tube further comprises polytetrafluoroethylene.

5C. The tube of embodiment 4C, wherein the polyoxymethylene homopolymer and polytetrafluoroethylene are each melted and then uniformly blended together.

6C. The tube of embodiment 4C, wherein the polyoxymethylene homopolymer is melted and polytetrafluoroethylene fibers are then uniformly blended with the polyoxymethylene homopolymer.

1D. The tube of any one of embodiments 1A, 1B and 1C-6C, the contact surface comprising a surface coating or material having low surface energy.

2D. The tube of embodiment 1D, the surface coating comprising a sol-gel coating.

3D. The tube of embodiment 1D, the surface coating comprising a hybrid organic-inorganic polymeric coating, optionally, ORMOCER® coating from Fraunhofer Institute, as described in the present application.

4D. The tube of embodiment 1D, wherein the surface coating or material includes a fluorinated polymer, optionally a member selected from the group consisting of: fluorinated ethylene propylene (FEP), ethylene tetrafluoroethylene (ETFE), polytetrafluoroethylene (PTFE), polychlorotrifluoroethylene (PCTFE) and perfluoroalkoxy (PFA).

1E. A method of feeding a gasket into a medical barrel, the method comprising:
providing a tube according to any one of embodiments 1A, 1B, 1C-6C and 1D-4D;
placing a gasket having an outer wall into the tube within the lumen such that compression of the outer wall against the contact surface retains the gasket in place within the lumen;
aligning the tube with a proximal open end of a medical barrel;
sliding the gasket through the tube such that the outer wall of the gasket contacts only the contact surface without contacting the entirety of the inner wall; and
transferring the gasket from the tube into the medical barrel.

2E. The method of embodiment 1E, the gasket comprising:
(a) a main body made of an elastic material and having a circumferential surface portion and an internal cavity, the cavity being defined by an inner surface portion of the gasket and being open-ended at one end;
(b) a film residing on at least a part of a circumferential outer portion of the gasket; and
(c) a plurality of non-continuous channels in or through the film being approximately parallel to the other non-continuous channels, each non-continuous channel of the plurality of non-continuous channels extending around the circumferential outer surface of the gasket and having a non-channel portion interrupting the non-continuous channel,
   wherein the non-channel portion of each non-continuous channel is positioned along the circumferential outer surface portion of the gasket such that it is not aligned with the non-channel portion of the immediately adjacent one or more non-continuous channels.

3E. The method of embodiment 2E, wherein the gasket is formed by a process for forming the plurality of non-continuous channels in or through the film residing on at least a part of the circumferential outer surface portion of the gasket, the process comprising the following steps:
(a) inserting a portion of one end of a mandrel into the open end of the cavity;
(b) positioning the mandrel and gasket in proximity to a laser; and
(c) applying laser beams emitted from the laser to a plurality of selected locations on a surface portion of the film while rotating the mandrel and the gasket along the mandrel's longitudinal axis to form the plurality of non-continuous channels in or through the film, each non-continuous channel being approximately parallel to the other non-continuous channels, each non-continuous channel of the plurality of non-continuous channels extending around the circumferential outer surface of the gasket and having a non-channel portion interrupting the non-continuous channel,
   wherein the non-channel portion of each non-continuous channel is positioned along the circumferential outer surface portion of the gasket such that it is not aligned with the non-channel portion of the immediately adjacent one or more non-continuous channels.

4E. The method of any one of embodiments 1E-3E, wherein the method is carried out without any flowable lubricant between the gasket and the tube.

While the invention has been described in detail and with reference to specific examples thereof, it will be apparent to one skilled in the art that various changes and modifications can be made therein without departing from the spirit and scope thereof.

1. A system for feeding a syringe gasket into a medical barrel, the system comprising:
a. a gasket configured for insertion into a medical barrel, the gasket comprising a main body made of an elastic material, the gasket having a distal nose cone configured to face a product stored within the medical barrel when inserted into the medical barrel and a circumferential surface portion extending proximally from the nose cone, at least a portion of the circumferential surface portion being configured to contact an interior sidewall of the medical barrel in compressive engagement; and
b. a tube for retaining the gasket and then feeding the gasket into the medical barrel, the tube comprising an elongate hollow tubular body comprising an internal lumen, the lumen having an inner wall which consists of a contact surface configured to contact a portion of the circumferential surface portion of the gasket and a non-contact surface occupying the remainder of the inner wall;
wherein the gasket is disposed within the tube such that, along a selected plane perpendicular to a central axis of the tube, a first part of the circumferential surface portion of the gasket contacts and is in compressive engagement with the contact surface while a second part of the circumferential surface portion of the gasket either:
(i) is not in contact with the non-contact portion; or
(ii) is in contact with the non-contact portion but with less compression against the non-contact portion than exists between the first part of the circumferential surface portion of the gasket and the contact surface.

2. A system for feeding a syringe gasket into a medical barrel, the system comprising:
a. a gasket configured for insertion into a medical barrel, the gasket comprising a main body made of an elastic material, the gasket having a distal nose cone configured to face a product stored within the medical barrel when inserted into the medical barrel and a circumferential surface portion extending proximally from the nose cone, at least a portion of the circumferential surface portion being configured to contact an interior sidewall of the medical barrel in compressive engagement; and
b. a tube for retaining the gasket and then feeding the gasket into the medical barrel, the tube comprising an elongate hollow tubular body comprising an internal lumen, the lumen having an inner wall which consists of a contact surface configured to contact a portion of the circumferential surface portion of the gasket and a non-contact surface occupying the remainder of the inner wall, the contact surface comprising two or more guide rails extending axially along the lumen;
wherein the gasket is disposed within the tube such that, along a selected plane perpendicular to a central axis of the tube, a first part of the circumferential surface portion of the gasket contacts and is in compressive engagement with the two or more guide rails while a second part of the circumferential surface portion of the gasket either:
(i) is not in contact with the non-contact portion; or
(ii) is in contact with the non-contact portion but with less compression against the non-contact portion than exists between the first part of the circumferential surface portion of the gasket and the two or more guide rails.

3. A method for feeding a syringe gasket into a medical barrel, the method comprising:
a. providing a gasket configured for insertion into a medical barrel, the gasket comprising a main body made of an elastic material, the gasket having a distal nose cone configured to face a product stored within the medical barrel when inserted into the medical barrel and a circumferential surface portion extending proximally from the nose cone, at least a portion of the circumferential surface portion being configured to contact an interior sidewall of the medical barrel in compressive engagement;
b. providing a tube for retaining the gasket and then feeding the gasket into the medical barrel, the tube comprising an elongate hollow tubular body comprising an internal lumen, the lumen having an inner wall which consists of a contact surface configured to contact a portion of the circumferential surface portion of the gasket and a non-contact surface occupying the remainder of the inner wall;
c. placing the gasket into the tube such that, along a selected plane perpendicular to a central axis of the tube, a first part of the circumferential surface portion of the gasket contacts and is in compressive engagement with the contact surface while a second part of the circumferential surface portion of the gasket either:
(i) is not in contact with the non-contact portion; or
(ii) is in contact with the non-contact portion but with less compression against the non-contact portion than exists between the first part of the circumferential surface portion of the gasket and the contact surface.
d. aligning the tube with a proximal open end of the medical barrel;
e. sliding the gasket through the tube, wherein contact and compression between the circumferential surface portion of the gasket and the inner wall of the lumen as recited in step (c) is maintained as the gasket slides through the tube; and
f. transferring the gasket from the tube into the medical barrel.

4. A method for feeding a syringe gasket into a medical barrel, the method comprising:
a. providing a gasket configured for insertion into a medical barrel, the gasket comprising a main body made of an elastic material, the gasket having a distal nose cone configured to face a product stored within the medical barrel when inserted into the medical barrel and a circumferential surface portion extending proximally from the nose cone, at least a portion of the circumferential surface portion being configured to contact an interior sidewall of the medical barrel in compressive engagement;
b. providing a tube for retaining the gasket and then feeding the gasket into the medical barrel, the tube comprising an elongate hollow tubular body comprising an internal lumen, the lumen having an inner wall which consists of a contact surface configured to contact a portion of the circumferential surface portion of the gasket and a non-contact surface occupying the remainder of the inner wall, the contact surface comprising two or more guide rails extending axially along the lumen;
c. placing the gasket into the tube such that, along a selected plane perpendicular to a central axis of the tube, a first part of the circumferential surface portion of the gasket contacts and is in compressive engagement with the two or more guide rails while a second part of the circumferential surface portion of the gasket either:
(i) is not in contact with the non-contact portion; or
(ii) is in contact with the non-contact portion but with less compression against the non-contact portion than exists between the first part of the circumferential surface portion of the gasket and the two or more guide rails;
d. aligning the tube with a proximal open end of the medical barrel;
e. sliding the gasket through the tube, wherein contact and compression between the circumferential surface portion of the gasket and the inner wall of the lumen as recited in step (c) is maintained as the gasket slides through the tube; and
f. transferring the gasket from the tube into the medical barrel.

5. The method of claim 4, wherein the contact surface has a surface area which is less than that of the inner wall.

6. The method of claim 4, wherein the guiderails are outwardly rounded along a cross-sectional plane.

7. The method of claim 4, wherein the guiderails are inwardly rounded along a cross-sectional plane.

8. The method of claim 4, comprising two to eight guiderails.

9. The method of claim 4, comprising an even number of guiderails, wherein each guiderail opposes another guiderail located 180 degrees from it around the lumen.

10. The system of claim 2, further comprising a medical barrel with a proximal end thereof aligned with the tube and, in this position, configured to receive the gasket from the tube.

11. The method of claim 4, the contact surface comprising a surface coating having low surface energy.

12. The method of claim 11, the surface coating comprising a sol-gel coating.

13. The method of claim 11, the surface coating comprising an inorganic-organic hydride polymer.

14. The method of claim 13, the surface coating comprising:
a. a hydrolytic condensate, prepared from a silane of the formula RₘSiX₄₋ₘ having the following meanings:
R=crosslinkable organic radical
X=hydrolyzable and condensable group
m=1 or 2 or 3
with a metal compound;
b. a prepolymer that is crosslinkable with the radicals R of the silane,
c. one or more non-crosslinkable organofunctional silane(s); and
d. a low-volatility oxide.

15. The method of claim 11, wherein the surface coating includes a fluorinated polymer which is a member selected from the group consisting of: flourinated ethylene propylene (FEP), ethylene tetrafluoroethylene (ETFE), polytetrafluoroethylene (PTFE), polychlorotrifluoroethylene (PCTFE) and perfluoroalkoxy (PFA).

16. The method of claim 4, wherein the tube is composed of polyoxymethylene homopolymer, polytetrafluoroethylene or a melt blend of the two.

17. The method of claim 4, wherein the tube is composed of polyoxymethylene homopolymer with polytetrafluoroethylene fibers uniformly blended in the polyoxymethylene homopolymer.

18. The method of claim 4, the circumferential surface portion of the gasket comprising one or more ribs that protrude radially outward and are configured to engage and provide a seal between the gasket and a medical barrel into which the gasket is disposed, wherein at least one of the one or more ribs comprises a channel or channels running along at least a portion of the circumference of the at least one of the one or more ribs.

19. The method of claim 18, wherein the one or more ribs comprises a distal rib adjacent to the nose cone, the channel or channels being provided on the distal rib.

20. The method of claim 18, wherein the channel or channels are laser cut.

21. The method of claim 18, wherein the channel or channels are non-continuous.

22. The method of claim 4, comprising a film residing on at least a part of the circumferential outer portion of the gasket.

23. The method of claim 4, the gasket comprising:
a. the main body comprising an internal cavity, the cavity being defined by an inner surface portion of the gasket and being open-ended at one end;
b. a film residing on at least a part of the circumferential surface portion of the gasket; and
c. a plurality of non-continuous channels in or through the film being approximately parallel to the other non-continuous channels, each non-continuous channel of the plurality of non-continuous channels extending around the circumferential outer surface of the gasket and having a non-channel portion interrupting the non-continuous channel,
wherein the non-channel portion of each non-continuous channel is positioned along the circumferential outer surface portion of the gasket such that it is not aligned with the non-channel portion of the immediately adjacent non-continuous channel.

24. The method of claim 23, wherein the plurality of non-continuous channels are provided on a rib of the gasket.

25. The system or method of claim 4, wherein there is no flowable lubricant in the tube.

26. The method of claim 24, wherein the rib is a distal rib adjacent to the nose cone.
